(19) United States
(12) Patent Application Publication
(10) Pub. No.: US 2016/0184162 A1
(43) Pub. Date: Jun. 30, 2016

(54) COMBINED SONIC AND HEAT SKIN CARE DEVICE

(71) Applicant: L'Oreal, Paris (FR)

(72) Inventors: Joseph W. Grez, North Bend, WA (US); Zane Bowman Allen Miller, Seattle, WA (US); Vincenzo Casasanta, III, Woodinville, WA (US); James Christopher McInnes, Seattle, WA (US)

(73) Assignee: L'Oreal, Paris (FR)

(21) Appl. No.: 14/586,490

(22) Filed: Dec. 30, 2014

(51) Int. Cl. A61H 1/00; A61F 7/00

(52) U.S. Cl. CPC A61H 1/008; A61F 7/00

(57) ABSTRACT

An applicator for use in treating a portion of skin includes an applicator tip that has an end configured to contact the portion of skin, a sonic source mechanically coupled to the applicator tip, a heat source thermally coupled to an end of the applicator tip, and a controller. The applicator tip includes a thermally-conductive material. During operation of the applicator, the controller controls operation of the sonic source to impart mechanical movements to the applicator tip at a sonic frequency and controls operation of the heat source to transfer heat to the end of the applicator tip via the thermally-conductive material of the applicator tip.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with an aspect of the present disclosure, an applicator for use in treating a portion of skin includes an applicator tip having an end configured to contact the portion of skin, a sonic source mechanically coupled to the applicator tip, a heat source thermally coupled to the end of the applicator tip, and at least one controller configured to control operation of the sonic source and the heat source. The end of the applicator tip includes a thermally-conductive material. During operation of the applicator, the at least one controller controls operation of the sonic source to impart mechanical movements to the applicator tip at a sonic frequency and controls operation of the heat source to transfer heat to the end of the applicator tip via the thermally-conductive material of the applicator tip.

In one example, the heat source includes a heating disk. In another example the heating disk includes a plurality of layers of electrical components positioned on an alumina substrate. In another example, the applicator includes a heat sensor configured to generate a signal based on a temperature of the applicator tip and to send the signal to the controller. In another example, the temperature of the applicator tip includes one or more of a temperature of the applicator tip or a temperature of the heat source. In another example, the at least one controller is configured to control the heat source based on the signal sent to the at least one controller by the heat sensor.

In another example, the at least one controller is configured to control operation of the heat source based on a target temperature of the end of the applicator tip. In another example, the target temperature is in a range from about 32° C. to about 50° C. In another example, the mechanical movements at the sonic frequency are reciprocating mechanical movements. In another example, the mechanical movements at the sonic frequency are oscillatory mechanical movements.

In another example, the applicator further includes a contact member around the end of the applicator tip where the contact member is configured to contact the portion of skin and where the contact member and the end of the applicator tip form a concave pocket configured to contain formulation between the end of the applicator tip and the portion of skin. In another example, the end of the applicator tip is formed with a concave shape, and wherein the concave shape forms a pocket configured to contain formulation between the end of the applicator tip and the portion of skin. In another example, the thermally-conductive material forms the concave shape of the end of the applicator tip. In another example, the applicator tip includes a thermally-insulating material, and wherein the thermally-conductive material is embedded within the thermally-insulating material.

In another embodiment, a method is used for treating a portion of skin using an applicator that includes an applicator tip where the applicator tip includes an end configured to contact the portion of skin. The method includes activating a sonic source of the applicator where the sonic source is mechanically coupled to the applicator tip, activating a heat source of the applicator where the heat source is thermally coupled to the end of the applicator tip, controlling operation of the sonic source to impart mechanical movements to the applicator tip at a sonic frequency, and controlling operation of the heat source to transfer heat to the end of the applicator tip via a thermally-conductive material of the applicator tip.

In one example, the method further includes applying a treatment formulation between the end of the applicator tip and the portion of skin during operation of the sonic source and operation of the heat source. In another example, applying the treatment formulation includes bringing the end of the applicator tip into contact with the treatment formulation when the treatment formulation is in a non-fluid form. In another example, the operation of the heat source is controlled such that heat transferred to the end of the applicator tip causes the treatment formulation to convert from the non-fluid form to a fluid form when the treatment formulation is in contact with the end of the applicator tip. In another example, applying the treatment formulation includes applying the treatment formulation in the fluid form to the portion of skin. In another example, the mechanical movements imparted to the applicator tip at the sonic frequency apply a shear stress to the treatment formulation that causes a viscosity of the treatment formulation to be lowered.

### DESCRIPTION OF THE DRAWINGS

The foregoing aspects and many of the attendant advantages of the subject matter disclosed herein will become more readily appreciated as the same become better understood by reference to the following detailed description, when taken in conjunction with the accompanying drawings, wherein:

FIG. 1A depicts a cross-sectional view of one embodiment of an applicator with a reciprocating tip in accordance with aspects of the present disclosure;

FIG. 1B depicts a cross-sectional view of one embodiment of an applicator with an oscillating tip in accordance with aspects of the present disclosure;

FIG. 2 depicts an embodiment of a reciprocating applicator in accordance with aspects of the present disclosure;

FIG. 3 depicts an embodiment of an oscillating applicator in accordance with aspects of the present disclosure;

FIG. 4 depicts a thermogram chart showing examples of the effect of heat on skin to influence phospholipid transitions within the skin in accordance with aspects of the disclosed embodiments;

FIG. 5 depicts a chart with a series of example shear rate curves demonstrating a decrease in viscosity with increased temperature in accordance with aspects of the disclosed embodiments;

FIG. 6 depicts a chart showing the effect on delivery of a treatment formulation using a sonically-activated tip at various temperatures in accordance with aspects of the disclosed embodiments;

FIG. 7 depicts a chart showing the influence of applicator tip material hardness to delivery of an ingredient from a treatment formulation into the skin in accordance with aspects of the disclosed embodiments;

FIGS. 8A and 8B depict charts indicating some of the benefits of applying treatment formulation using a heated, sonically-activated applicator tip instead of a sonically-activated applicator tip without heat in accordance with aspects of the disclosed embodiments;

FIGS. 9A and 9B depict charts showing evidence of consumers' increased pleasant experience using a heated, sonically-activated applicator tip over a sonically-activated applicator tip without heat in accordance with aspects of the disclosed embodiments;

FIGS. 10A to 10C depict an embodiment of an applicator tip for use with a heated, sonically-activated application process in accordance with aspects of the disclosed embodiments; and

FIGS. 11A to 11C depict a process of converting a treatment formulation from a non-fluid form to a fluid form and applying the cosmetic formation to a portion of skin in fluid form in accordance with aspects of the disclosed embodiments.

### DETAILED DESCRIPTION

The detailed description set forth below in connection with the appended drawings where like numerals reference like elements is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the claimed subject matter to the precise forms disclosed.

The following discussion provides examples of systems, apparatuses, and/or methods for implementing technologies and methodologies for treating a portion of skin using a combination of heat and mechanical movement at a sonic frequency. In an embodiment, an applicator includes an applicator tip that has an end configured to contact the portion of skin, a sonic source mechanically coupled to the applicator tip, a heat source thermally coupled to an end of the applicator tip, and a controller. The applicator tip includes a thermally-conductive material. During operation of the applicator, the controller controls operation of the sonic source to impart mechanical movements to the applicator tip at a sonic frequency and controls operation of the heat source to transfer heat to the end of the applicator tip via the thermally-conductive material of the applicator tip. In another embodiment, a method includes activating a sonic source of the applicator that is mechanically coupled to the applicator tip, activating a heat source of the applicator that is thermally coupled to the end of the applicator tip, controlling operation of the sonic source to impart mechanical movements to the applicator tip at a sonic frequency, and controlling operation of the heat source to transfer heat to the end of the applicator tip via a thermally-conductive material of the applicator tip.

In the following description, numerous specific details are set forth in order to provide a thorough understanding of one or more embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

Many consumers desire smooth and youthful skin with an even tone. Such characteristics can be achieved by use of treatment formulations. As used herein, the term “treatment formulation” refers to makeup, personal soaps, skin care products, hair care products, or any other cosmetic product. Many active ingredients for treatment formulations have been identified as being able to address these and other consumer skin care desires (e.g., active ingredients for anti-aging, lightening, etc.). However, skin is an effective barrier to many active ingredients. When treatment formulations are applied to skin, much of the active ingredient remains on the surface of the skin or is wiped away from the surface of the skin. Increasing absorption of active ingredients into skin will improve the effectiveness of treatment formulations for users.

In some embodiments, a treatment formulation is one or more of makeup, personal soap, skin care product, hair care product, or any other cosmetics product. In some examples, makeup includes foundation, blush, highlighter, bronzer, or any other type of makeup. In some examples, personal soap includes facial cleanser, body wash, or any other type of personal soap. In some examples, skin care products include lotions, skin exfoliants, masking formulations, or any other type of skin care product. In some examples, hair care products include shampoos, conditioners, shaving cream, or any other type of hair care products.

One way to increase absorption of active ingredients into skin is to apply treatment formulations using an applicator that has a sonic motion applicator tip. In one example, an applicator tip is configured to be moved in a reciprocating manner at a sonic frequency. In another example, an applicator tip is configured to be moved in an oscillatory manner at a sonic frequency. In some applications, sonic mechanical motion of an applicator tip increases adsorption of active ingredients in a number of ways, such as by loosening the outer layer of the skin (i.e., the stratum corneum).

Absorption of active ingredients into a portion of skin by sonic mechanical motion can be enhanced with the use of heat. Heating skin and/or treatment formulation enhances absorption of active ingredients in one or more ways, such as by altering phospholipid structures with the outer layer of the skin, by decreasing viscosity of the treatment formulation, by increasing delivery of the treatment formulation into the outer layer of the skin, and/or by reducing skin formations (e.g., crow's feet, fine lines, wrinkles, etc.) over extended periods of use.

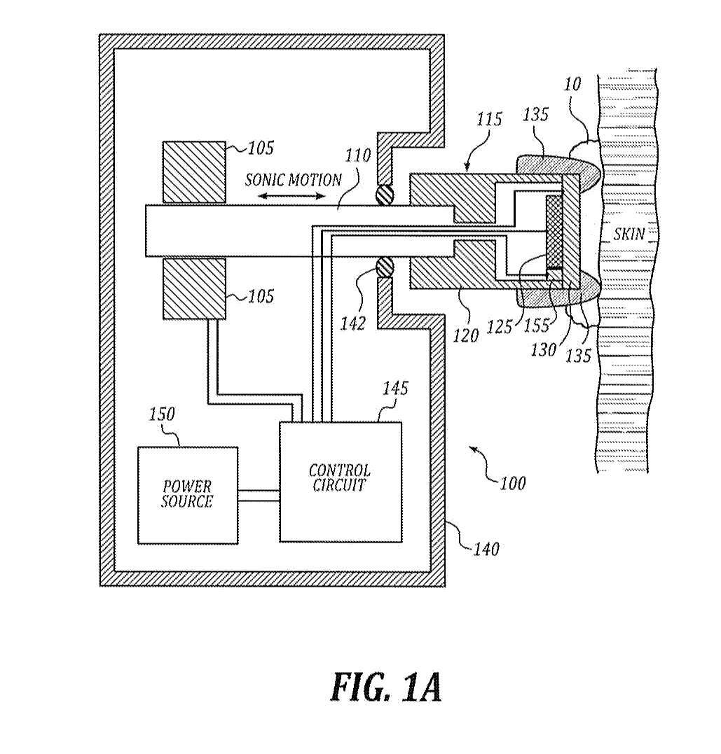

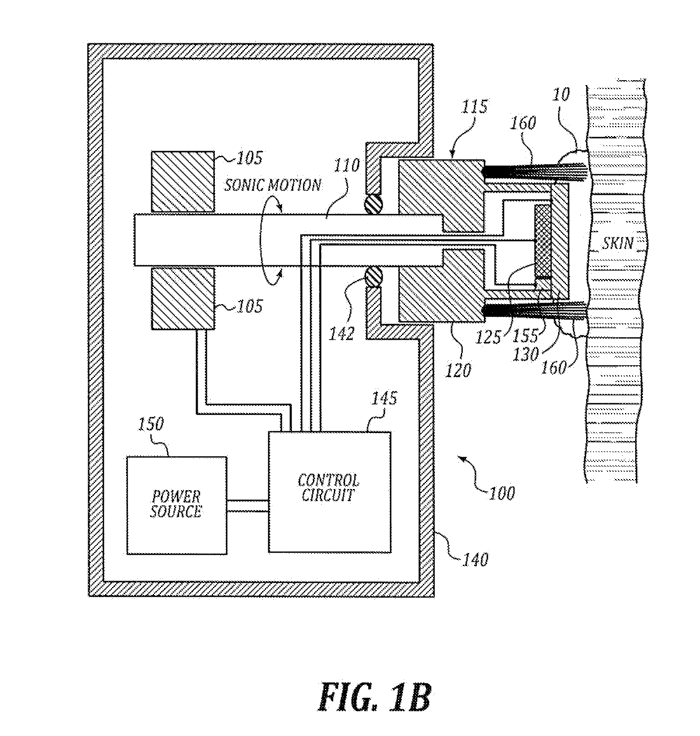

Turning now to FIG. 1A, there is shown one example illustrated in cross section of an applicator, generally designated 100, formed in accordance with aspects of the present disclosure. The applicator 100 includes a sonic source 105 (e.g., a motor) and a heat source 125 (i.e., a heating element). Both the sonic source 105 and the heat source 125 are directed toward a portion of skin at a common location. The sonic motion and heat provided by the device 100 effect transdermal delivery of a formulation 10 (e.g., a treatment formulation) into the portion of skin.

The heat source 125 generates heat. Heat generated by the heat source 125 is transferred to an end 130. In one embodiment, the end 130 is formed from a thermally-conductive material, such as a metal, a glass, or any other thermally-conductive material. In another embodiment, end 130 is formed from a thermally-insulating material (e.g., a plastic, graphite, a rubber, an epoxy, an elastomer, a gel, etc.) that is embedded with a thermally-conductive material (e.g., silver strands, carbon, etc.). The end 130 acts both as a barrier to isolate the heat source 125 and as a medium to transfer heat.

The heat source 125 can take a number of forms. In one embodiment, the heat source 125 comprises a heating disk. In one example, the heating disk comprises an alumina substrate with several layers of electrical components (e.g., resistors, insulators, and conductors). In one example, such electrical components are patterned on the substrate by thick film paste stencil printing. Thick film paste screen print processes offer a very low cost solution to pattern electrical components (e.g., resistor, conductors, and dielectrics) on ceramic substrates. Such heating disk heat sources are capable of supporting fast and controlled heating up to 100° C. In other embodiments, any number of other heat sources may be used to provide heat to the end 130.

The sonic source 105 provides reciprocating sonic motion to an applicator tip 115 via a shaft 110 that is operatively connected to the applicator tip 115. The applicator tip 115 optionally terminates in a concave contact member 135 configured to contact the portion of skin with a pocket suitable for containing formulation 10 therebetween. In another embodiment, not depicted in FIG. 1A, the contact member 135 is omitted and the end 130 is formed with a concave shape to create a pocket suitable for containing formulation 10 therebetween.

The sonic source 105 applies reciprocating sonic motion to the portion of skin via the contact member 135. In one embodiment, the contact member 135 is formed from one or more materials, such as an elastomeric material or a silicone rubber, that are soft enough to avoid discomfort or injury to the skin but firm enough to maintain its shape and impart sufficient sonic energy. Other exemplary materials can also be used, such as natural rubber, butyl rubber, and polyurethane. In certain embodiments, the soft contact member 135 improves transmission of the ultrasound by containing and moving the formulation 10 so as to facilitate contact between the applicator tip 115 and skin.

In one embodiment, operation of the sonic source 105 reciprocates the applicator tip 115 at an amplitude of 0.075 inch to 0.1 inch. In one embodiment, the sonic source 105 has a reciprocation rate of less than 1 kHz. In one embodiment, the sonic source 105 has a reciprocation rate of less than 200 Hz. In one embodiment, the sonic source 105 has a reciprocation rate of greater than 10 Hz.

The heat source 125 is contained within a housing 120 of the applicator tip 115. In the illustrated embodiment, the placement of the heat source 125 is on the central axis of the shaft 110 and applicator tip 115. However, it will be appreciated that other arrangements are also contemplated.

In the embodiment of the applicator 100 shown in FIG. 1A, a housing 140 provides a grip/handle for a user of the applicator 100. The housing 140 contains the sonic source 105 and partially encloses the shaft 110, which passes from the housing 140 towards the applicator tip 115 via an aperture fitted with a gasket 142 configured to allow reciprocating motion of the shaft 110 while sealing the inner chamber of the housing 140.

The housing 140 further contains a control circuit 145 (e.g., a printed circuit board, field-programmable gate array, ASIC, etc.) configured to operatively control the sonic source 105 and the heat source 125. The control circuit 145 controls the sonic source 105 and the heat source 125 by sending signals to the sonic source 105 and the heat source 125. In one embodiment, the control circuit 145 includes a single circuit that controls the sonic source 105 and the heat source 125. In another embodiment, the control circuit 145 includes a single circuit to control the sonic source 105 and a single circuit to control the heat source 125. In other embodiments, the control circuit 145 includes other combinations of a number of circuits. A power source 150 is contained within the housing 140 and powers the control circuit 145, the sonic source 105, and the heat source 125. Power requirements of the power source 140 will depend on the desired function of the device 100. The tip 115 includes a heat sensor 155 (e.g., a thermocouple, a thermistor, etc.) that is configured to generate a signal based on a temperature of the tip 115 (e.g., a temperature of the heat source 125 and/or a temperature of the end 130) and send the signal to the control circuit 145.

The control circuit 145 controls the heat source 125 based on the signal received from the heat sensor 155. In one embodiment, a target skin temperature is less than 50° C. with a preferred temperature near about 40° C. In one embodiment, the applicator tip maintains a steady temperature during use. Normal skin temperature is typically around 32° C. The applicator tip is considered heated when it has a temperature above the normal skin temperature of the user. Room temperature varies depending on the location of the user, but is typically around 22° C. In certain circumstances, treatment formulations are at room temperature, whether in solid form or non-solid form, when first brought into contact with the portion of skin.

When the applicator tip is heated and placed into contact with the treatment formulation and/or the skin, the treatment formulation and/or skin will act as a heat sink and cause a reduction in the temperature of the applicator tip. During operation, as the applicator tip converts the treatment formulation from solid form to non-solid form or as the applicator tip is moved across the skin, the average temperature of the treatment formulation and/or skin may increase, thus reducing the effect of the treatment formulation and/or skin as a heat sink.

In one embodiment, the applicator tip is maintained at a substantially constant temperature. Maintaining a substantially constant temperature includes maintaining temperature within ±5% of a target temperature. Maintaining the applicator tip at a substantially constant temperature has benefits of safety for the consumer, better sensory experience for the consumer, and efficiency of heating. In one example, maintaining the applicator tip at a substantially constant temperature may be accomplished actively by at least one controller (e.g., control circuit 145) that monitors temperature of the applicator tip and adjusts power delivery to the heat source accordingly. In another example, maintaining the applicator tip at a substantially constant temperature may be accomplished passively by using a heat source that changes characteristics (e.g., resistance) based on its own temperature.

Another embodiment of applicator 100 is depicted in FIG. 1B. The applicator 100 depicted in FIG. 1B has similar components to the applicator 100 depicted in FIG. 1A. The sonic source 105 provides oscillating sonic motion to the applicator tip 115 via the shaft 110. The applicator tip 115 depicted in FIG. 1B includes bristles 160 located around the end 130. The bristles 160 are configured to contact the portion of skin with a pocket suitable for containing formulation 10 therebetween.

In one embodiment, operation of the sonic source 105 in FIG. 1B oscillates the applicator tip 115 at an amplitude from about 2° to about 7°. In one embodiment, the sonic source 105 has an oscillation rate of less than 1 kHz. In one embodiment, the sonic source 105 has an oscillation rate of less than 200 Hz. In one embodiment, the sonic source 105 has an oscillation rate of greater than 10 Hz. Various frequencies and strains of sonic oscillating motion are described in U.S. Pat. No. 7,320,691, which is incorporated herein by reference in its entirety. In another embodiment, the sonic source 105 oscillates the applicator tip 115 at frequencies from about 80 Hz to about 200 Hz and a duty cycle of about 38-44%. In other examples, operational frequencies and oscillation amplitudes of applicators are varied depending in part on an intended application and/or characteristics of the applicator tip, such as its inertial properties, etc. In an embodiment of the present disclosure, the frequency ranges are selected so as to drive the attached head at near resonance. Thus, selected frequency ranges are dependent, in part, on the inertial properties of the attached head. It will be appreciated that driving the attached head at near resonance provides many benefits, including the ability to drive the attached head at suitable amplitudes in loaded conditions (e.g., when contacting the skin). For a more detailed discussion on the design parameters of the appliance, please see U.S. Pat. No. 7,786,646.

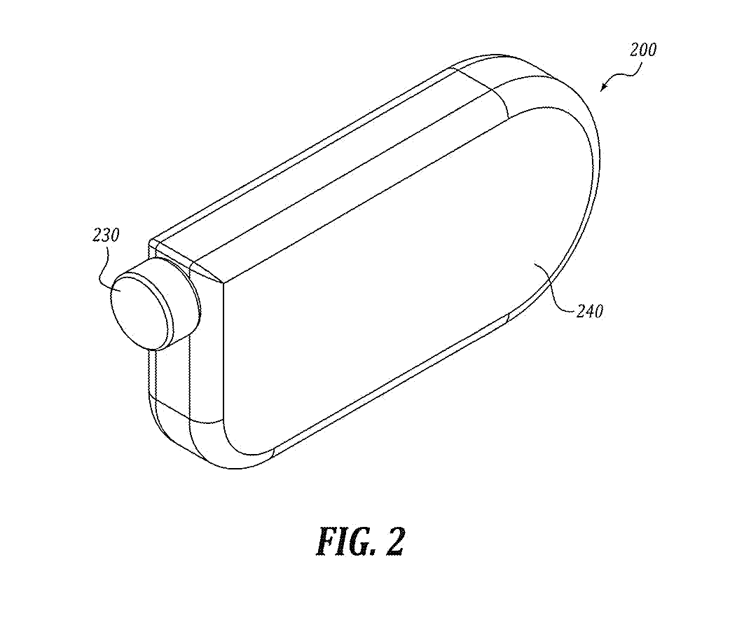

One embodiment of an applicator with a reciprocating sonic tip is the OPAL (produced by Clarisonic of Redmond, Wash.), which is illustrated in a form modified from the commercial device, in FIG. 2. In this applicator 200, the tip 230 creates strain on a portion of skin immediately adjacent to the area of the portion of skin that is in contact with the tip 230 of the applicator 200. An exemplary reciprocating applicator (such as the OPAL) that can apply a reciprocating sonic motion through a device head (e.g., tip 230) is described in U.S. Patent Application Publication No. 2009/0306577, which is incorporated herein by reference in its entirety. This action increases skin permeability by temporarily flexing and enlarging dermatoglyphs, paracellular spaces or transappendageal pathways such as hair follicles and sweat glands, which in turn increases dermal delivery. The action of the tip 230, which is substantially perpendicular to the skin, also acts to drive a formulation into the epidermis. This driving force occurs regardless of the formulation composition. The applicator 200 includes a body 240 that allows a user of the applicator 200 to hold the applicator 200 with the tip 230 impacting a portion of skin.

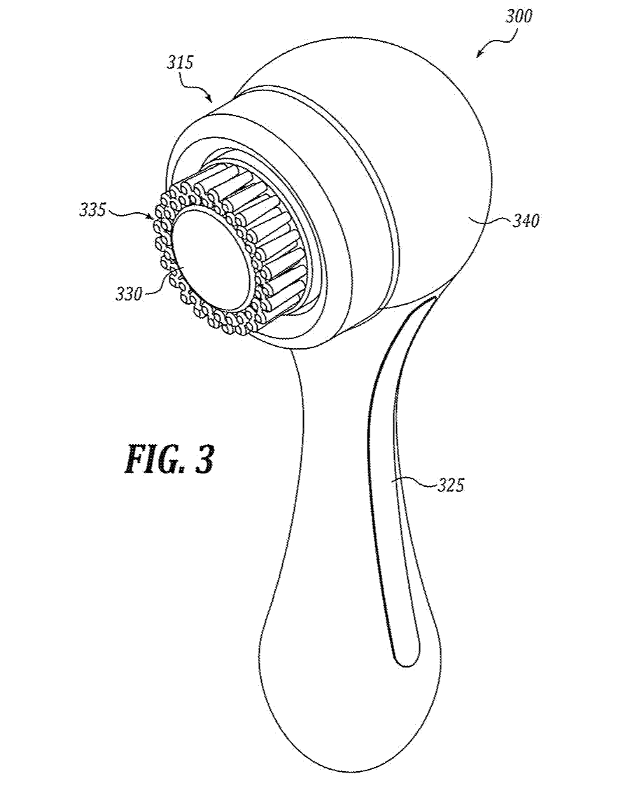

One embodiment of an applicator with an oscillating sonic tip is the CLARISONIC BRUSH (Clarisonic, Redmond, Wash.), which is illustrated in a form modified from the commercial device, in FIG. 3. In the applicator 300, a brush head 315 is releasably coupled to a body 340 that includes a motor or other source of oscillatory motion. The motor or other source of oscillatory motion is translated to the brush head 315. The brush head 315 includes a bristle field 335 surrounding a tip 330 in such a way that skin strain occurs in or immediately adjacent to the area of the skin that is in contact with the bristle field 335 and/or tip 330. In one embodiment, the bristle field 335 is omitted such that the tip 330 contacts the portion of skin without any bristles contacting the portion of skin. In one example, when the bristle field 335 is omitted, the tip 330 has a concave shape that creates a pocket suitable for containing formulation between the tip 330 and the portion of skin.

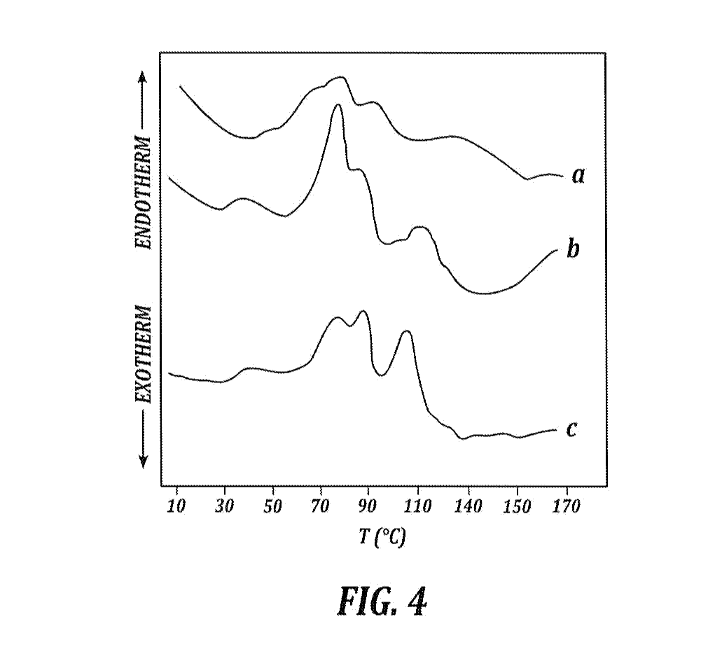

Any number of advantages are gained by using an applicator tip operating with sonic motion (hereinafter a “sonically-activated applicator tip”) that is also heated. FIG. 4 depicts a calorimetry thermogram chart showing the thermal transitions of the intercellular lipid layers of the stratum corneum at various levels of hydration. As shown in FIG. 4, sharp transitions occur above about 65° C. However, that temperature exceeds comfort and can result in skin damage (e.g., burns) with prolonged contact. FIG. 4 also depicts a smaller transition between about 35° C. and about 45° C. Temperatures in this range are within a typical comfort and safety range for application to the skin. The lipid structural transitions in this temperature range are associated with greater permeability of molecules through the stratum corneum as compared to room temperature. The advantage of increasing the skin temperature and thus altering the intercellular lipid structure is that it may allow active ingredients an opportunity to diffuse further into the skin than when the skin is at a normal temperature.

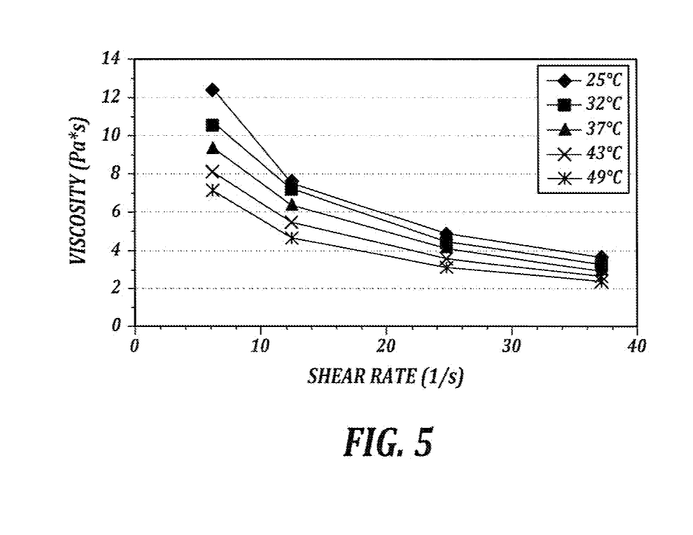

FIG. 5 depicts a chart with a series of example shear rate curves demonstrating a decrease in viscosity with increased temperature within a range of 25° C. to 49° C. A decrease in fluid viscosity with an increase in temperature is common in fluids that do not undergo a phase change with temperature. Decreasing the viscosity of a treatment formulation has the advantage that the treatment formulation may flow more readily upon the skin surface and potentially reach deeper into skin surface features (e.g., dermatoglyphs, pores, hair follicles, fine lines, wrinkles, etc.). These effects leave more formulation on the surface of the skin and available for subsequent absorption.

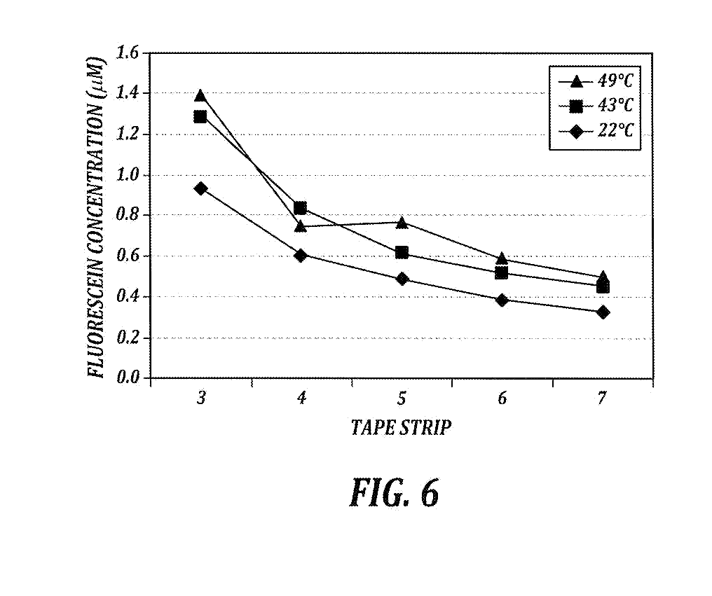

FIG. 6 depicts a chart showing the effect on delivery of a treatment formulation using a sonically-activated tip at various temperatures. A treatment formulation with added fluorescein was applied to porcine skin with a sonically-activated applicator tip at various temperatures. More specifically, the sonically-activated applicator tip applied the treatment formulation with added fluorescein at 22° C. (about room temperature), at 43° C., and at 49° C.

The graph in FIG. 6 shows the fluorescein concentration (n=20 per data point) at increasing depth within the skin as assayed with tape strips (higher strip numbers indicates deeper skin layers). As shown in the graph, the elevated temperatures at 43° C. and at 49° C. resulted in increased concentration of the fluorescein in the skin over the concentration of the fluorescein in the skin when applied at 22° C. The difference between the 22° C. temperature curve and the 43° C. and 49° C. temperature curves are statistically significant (p<0.05).

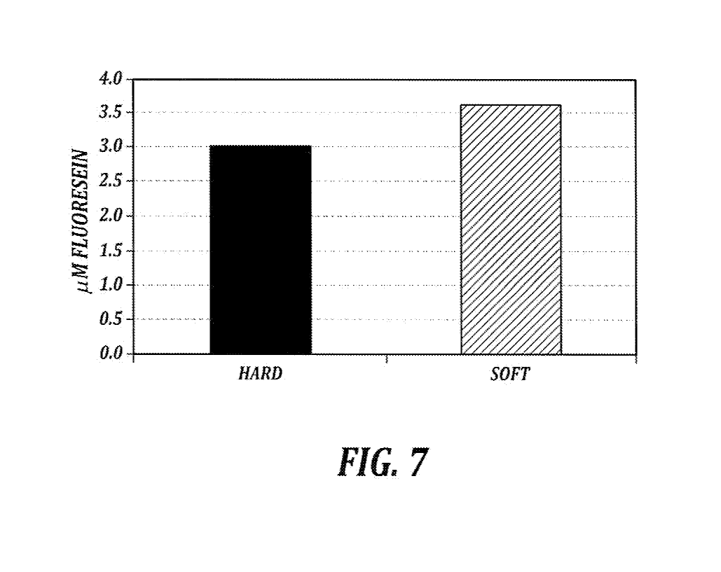

FIG. 7 depicts a chart showing the influence of applicator tip material hardness to delivery of an ingredient from a treatment formulation into the skin. Two different applicator tips were used while all other treatment variables were held constant. The two applicator tips include a “hard” applicator tip and a “soft” applicator tip. A hard applicator tip is an applicator tip that is rigid at all locations that touch the skin. A soft applicator tip is an applicator tip that is not rigid at all locations that touch the skin. In the particular investigation represented in FIG. 7, the hard applicator tip was a ceramic disk and the soft applicator tip utilized the same ceramic disk having a ring of deformable silicone material. The center of the deformable silicone material left the ceramic disk and the center formed a pocket for the treatment formulation. The pocket of treatment formulation allowed the soft applicator tip to glide more easily across the skin than the hard applicator tip. As shown in FIG. 7, the soft applicator tip delivered more treatment formulation (as measured by an amount of dye within the cosmetic formation) to the skin. Statistical testing indicated a significant difference between treatments (p=0.03).

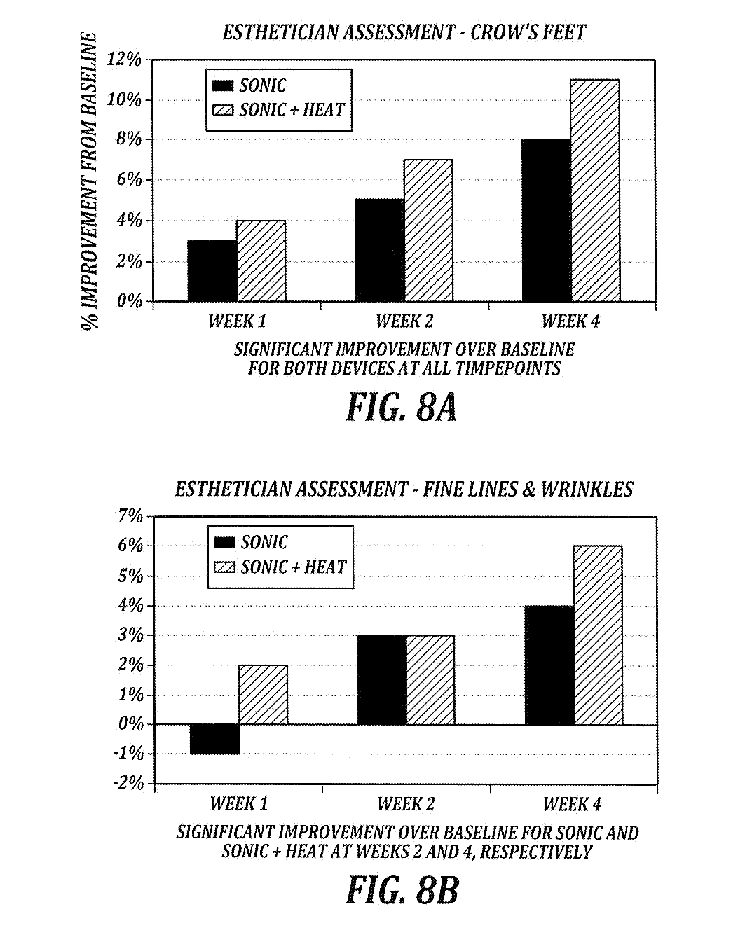

FIGS. 8A and 8B depict charts indicating some of the benefits of applying cosmetic using a heated, sonically-activated applicator tip instead of a sonically-activated applicator tip without heat. The data for the charts in FIGS. 8A and 8B was obtained from a study that conducted in a 4-week, randomized, split-face study with eleven subjects. The treatment formulation applied with the treatments was a glycoserum with proxylane. Assessments of clinical changes in crow's feet and fine lines and wrinkles in the skin surrounding the eye were made by an esthetician.

As shown in FIG. 8A, both the treatment using a sonically-activated applicator tip without heat and the treatment using a heated, sonically-activated applicator tip resulted in a reduction in crow's feet over the study. Although there was no significant difference between treatments for this study, the tendency was for the heated, sonically-activated applicator tip treatment to result in a larger reduction at each time point. As shown in FIG. 8B, the heated, sonically-activated applicator tip treatment resulted in a reduction in fine lines and wrinkles from baseline at each time point. The sonically-activated applicator tip without heat at resulted in a reduction in fine lines and wrinkles from baseline the 2-week and the 4-week time points.

One difficulty with treating skin conditions using treatment formulations is the amount of time required for consumers to visibly see the benefits. Because skin is an effective barrier to many active ingredients of treatment formulations, those active ingredients diffuse slowly into the skin. The result is that it can take a number of days or weeks for the visible benefits of treatment formulation treatment to appear. Consumers can become frustrated with the slow progress and lack of results in the short-term and abandon treatment formulation treatments before the benefits are visible.

Using a heated, sonically-activated applicator tip for application of treatment formulation can increase the time that consumers are willing to continue using the treatment formulation without seeing visible benefits. Many consumers find the sensation of a sonically-activated applicator tip to be pleasant. Adding gentle heat (i.e., heat that does not injure or damage the skin) to the applicator tip can enhance the consumer's experience using the sonically-activated applicator tip. Consumers are more likely to continue using an applicator tip without seeing visible benefits if the consumers find the use of applicator tips to be pleasant. If consumers continue using a heated, sonically-activated applicator tip for the experience alone, consumers may continue using treatment formulations for sufficient time to allow the benefits to be realized.

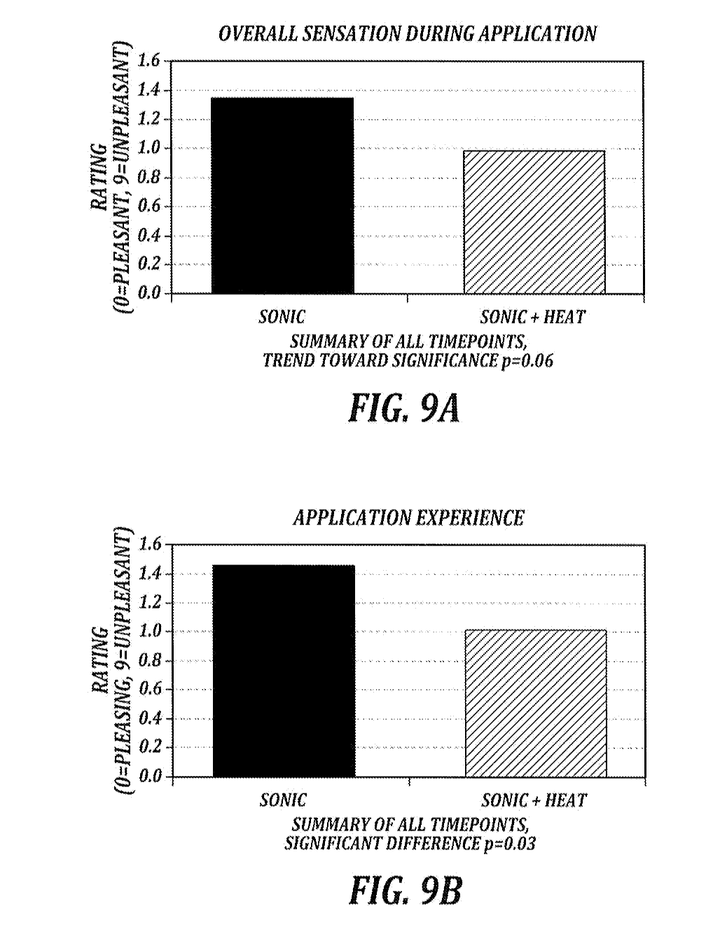

FIGS. 9A and 9B depict charts showing evidence of consumers' increased pleasant experience using a heated, sonically-activated applicator tip over a sonically-activated applicator tip without heat. A four-week evaluation was conducted in which a sonically-activated applicator tip without heat and a heated, sonically-activated applicator tip were applied to the skin around the eyes of eleven subjects. Subjects were asked weekly to evaluate the sensation in the treated area during application and their application experience on a scale of 0 (pleasing) to 9 (unpleasant).

FIG. 9A depicts subjects' responses of the sensation in the treated area, with 0 being the most pleasant experience and 9 being an unpleasant experience. Subjects rated the sensation of the sonically-activated applicator tip without heat to be 1.34 and the sensation of the heated, sonically-activated applicator tip to be 0.98. Thus, the subjects found the sensation of the heated, sonically-activated applicator tip to be more pleasant than the sensation of the sonically-activated applicator tip without heat.

FIG. 9B depicts subjects' responses of the sensation of the total application experience, with 0 being the most pleasant experience and 9 being an unpleasant experience. Subjects rated the total application experience of the sonically-activated applicator tip without heat to be 1.47 and the total application experience of the heated, sonically-activated applicator tip to be 1.05. Thus, the subjects found the application experience of the heated, sonically-activated applicator tip to be more pleasing than the application experience of the sonically-activated applicator tip without heat. In addition to the evaluation reflected in FIGS. 9A and 9B, investigation was done with the heated, sonically-activated applicator tip to gain sensory input into heat applied to the skin. In general, a temperature of 40±1° C. was found to be tolerable on the skin surface if the heated, sonically-activated applicator tip was continuously moving across the skin surface. Tolerance of the heated, sonically-activated applicator tip decreased as the device was held in one place and/or the heated, sonically-activated applicator tip applied temperatures above 42° C. Tolerance of the heated, sonically-activated applicator tip differed by region around the eyes, with areas within the periorbital region being less tolerant to the heat.

One difficulty with applying heat through a heated, sonically-activated applicator tip is to effectively transfer heat from the device to the skin while still delivering formulation and providing the consumer with a pleasurable sensation. Heat transferred from an object to the skin depends on a number of factors, such as the surface temperature of the object, the thermal properties of the object, and the contact time between the object and the skin. Heating skin may result in a burn which depends on both the temperature of the skin and duration that the temperature is held. Heating of the skin may be pleasurable at certain temperatures and short durations of exposure but becomes quickly non-pleasurable at higher temperatures and/or prolonged exposure. It is possible to generate heat using applicator tips that are made of rigid metal surfaces. However, such rigid metal surfaces are generally not desirable with moving applicators, such as with sonically-activated applicator tips. In contrast, soft and/or flexible materials, such as silicone, are generally more pleasant to the feel for consumers. However, soft and/or flexible materials typically have poor heat transfer characteristics.

Both goals of heat transfer and pleasurable consumer experience are accomplished using applicator tips that both transfer heat to the treatment formulation and/or the skin at target temperatures while simultaneously permitting the sonic motion that improves treatment formulation delivery and the sensorial experience preferred by consumers. For example, a soft and/or flexible material that conducts heat meets both of these goals. In some embodiments, the material of an applicator tip is a silicone rubber embedded with a thermally-conductive material, such as one or more of silver, carbon, or another material with high heat conductivity. The silicone rubber tip provides the pleasurable consumer experience and the embedded thermally-conductive material allows heat to be transferred to the portion of skin.

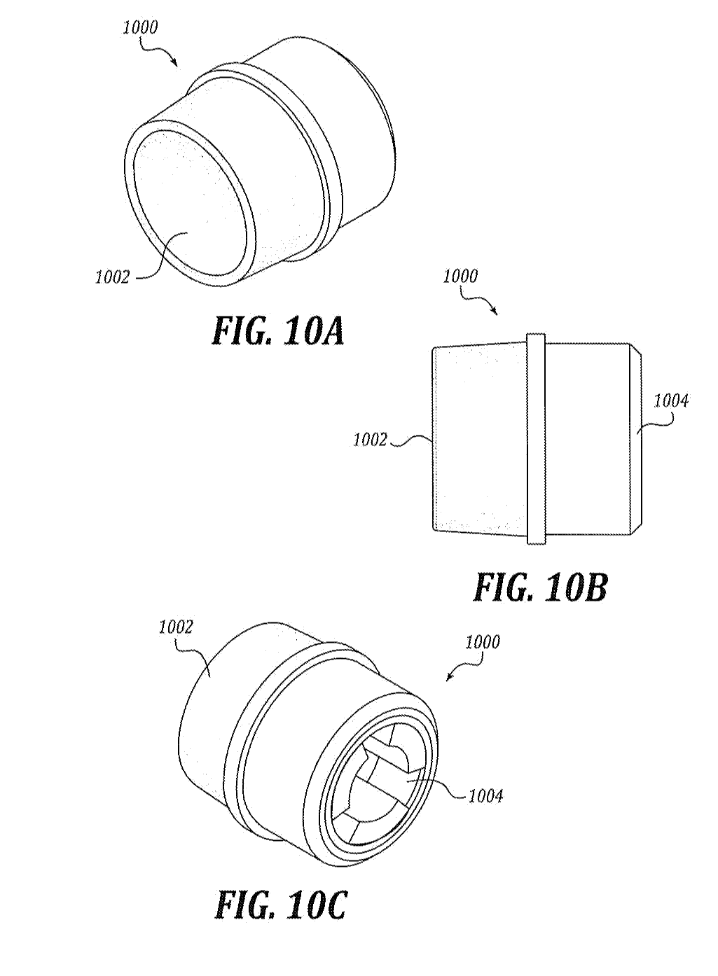

FIGS. 10A to 10C depict an embodiment of an applicator tip 1000 for use with a heated, sonically-activated application process. The applicator tip 1000 depicted in this particular embodiment includes an end 1002 that has a concave shape. In one embodiment, the end 1002 is made of a soft (e.g., non-rigid) material that provides a pleasant consumer experience when the applicator tip is sonically activated. The soft material also aids in delivery and infusion of a treatment formulation into the skin when the treatment formulation is applied by the applicator tip when the applicator tip is sonically activated. The end 1002 of the applicator tip 1000 has a concave cup shape that serves as a pocket for treatment formulation during application of the treatment formulation to a portion of skin. In one embodiment, the applicator tip is embedded with a thermally-conductive material, such as one or more of silver or carbon, that conducts heat during the application process. The heat conducted by the applicator tip is passed to the portion of skin to enhance the sensorial experience of the consumer and to aid in application of the treatment formulation. The applicator tip 1000 also has a coupling end 1004 that is configured to be coupled to a shaft of an applicator. In one embodiment, the coupling end 1004 includes one or more electrical connections that are configured to couple a control circuit in the applicator to a heat source and/or or temperature sensor in the applicator tip 1000.

Many treatment formulations, such as skin care products, are formulated in a liquid form, cream form, or gel form (collectively, a “fluid form”). Treatment formulations in fluid form are applied to the skin manually or with using an applicator device. While some treatment formulations are provided in fluid form in order to allow consumers to effectively apply the treatment formulations, it may be advantageous to provide some treatment formulations in a solid form, a semi-solid form, or a plastic form (collectively, a “non-fluid form”). Some advantages that can be gained by providing a treatment formulation in a non-fluid form include convenience in processing, convenience in packaging, efficient use, ingredient stability, or precise dosing. Other advantages may include providing a treatment formulation bound to a skin covering surface (e.g., a patch or a mask) or providing a treatment formulation that forms a skin covering surface itself. Still other advantages include that the formulation may have a component that is inactive as delivered to the skin but needs to be activated once on the skin. It would be advantageous for treatment formulations to be distributed in non-fluid form and then converted by consumers into a fluid form than can readily cover the surface of the skin and to be distributed evenly over the skin surface. A heated, sonically-activated applicator tip is capable of both converting treatment formulations from non-fluid form to fluid form and distributing treatment formulations in fluid form. The heated, sonically-activated applicator tip converts treatment formulations from non-fluid form to fluid form using either or both of heat and sonic movements. In one embodiment, heat also can convert treatment formulations from non-fluid form into a liquid form by melting the treatment formulations. For example, a treatment formulation in a non-fluid form has a melt temperature of about 40° C. When the treatment formulation is heated by heat transferred from the heated, sonically-activated applicator tip, the treatment formulation is converted to a fluid form. The heated, sonically-activated applicator tip is capable of applying the melted treatment formulation to the portion of skin. In another example, a treatment formulation in a non-fluid form includes a binder (e.g., wax) that holds the treatment formulation in non-fluid form. When heat is transferred from the heated, sonically-activated applicator tip to the treatment formulation, the melting binder releases (e.g., melts) and the treatment formulation in fluid form. In another embodiment, the treatment formulation has a shear thinning characteristic whereby shear stress applied to the treatment formulation lowers the viscosity of the treatment formulation, making the treatment formulation flow more easily. In this case, the sonic motion alone of the heated, sonically-activated applicator tip is capable of converting the treatment formulation from a non-fluid form to a fluid form. In another embodiment, a combination of heat and sonic motion from the heated, sonically-activated applicator tip converts a treatment formulation from a non-fluid form to a fluid form by heating the treatment formulation and applying a shear stress to the treatment formulation.

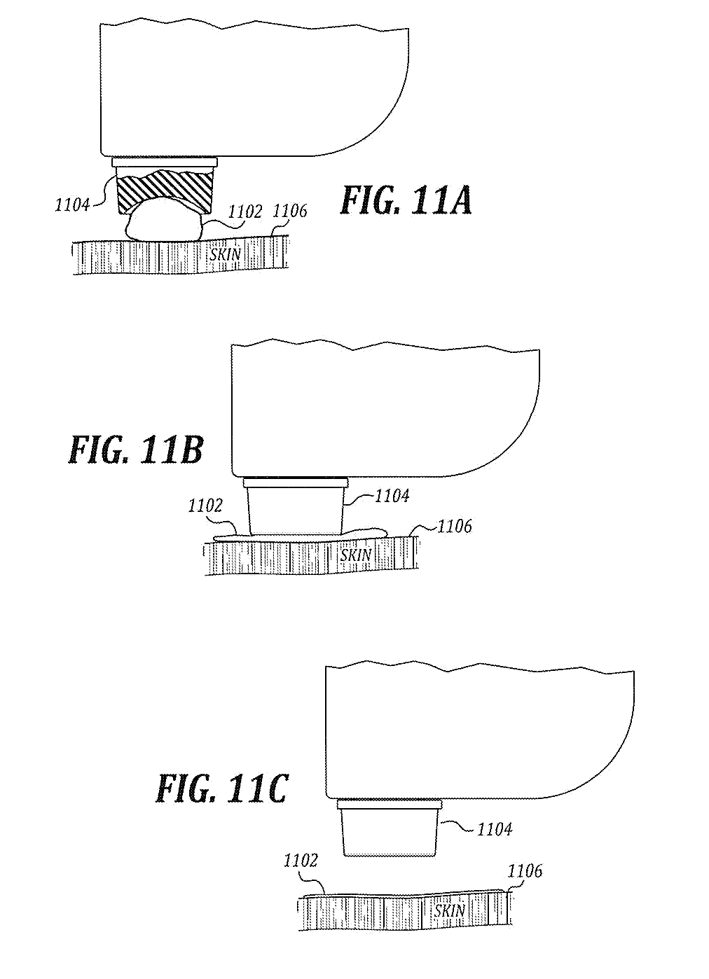

FIGS. 11A to 11C depict a process of converting a treatment formulation 1102 from a non-fluid form to a fluid form and applying the cosmetic formation to a portion of skin 1106 in fluid form. In FIG. 11A, the treatment formulation 1102 is placed on the portion of skin 1106 a non-fluid form. The treatment formulation is configured to be converted to a fluid form by an applicator 1104 with a heated, sonically-activated applicator tip. In FIG. 11B, the treatment formulation 1102 is converted from a non-fluid form to a fluid form by the applicator 1104 using the heated, sonically-activated applicator tip. The conversion of the treatment formulation 1102 from the non-fluid form to a fluid form is caused by either or both of heat transferred from the heated, sonically-activated applicator tip of the applicator 1104 to the treatment formulation 1102 or shear stress on the treatment formulation 1102 caused by sonic movements of the heated, sonically-activated applicator tip. In FIG. 11C, the treatment formulation 1102 in the fluid form has been applied to the portion of skin 1106.

During operation depicted in FIGS. 11B and 11C, from the time that the heated, sonically-activated applicator tip first engages the treatment formulation 1102 in the non-fluid form until the treatment formulation has been applied to the portion of skin 1106, the applicator 1104 is operated to cause mechanical motion of the applicator tip at a sonic frequency (e.g., sonic reciprocal movements or sonic oscillatory movements) and heat is transferred from to the sonically-activated applicator tip. Heat from the sonically-activated applicator tip is transferred to the treatment formulation 1102 and/or the portion of skin 1106. Either or both of the sonic mechanical motion and the transferred heat functions to convert the treatment formulation 1102 from the non-fluid form to the fluid form and to provide the consumer with the pleasant experience of sonic mechanical motion and heat where the treatment formulation is applied.

The methods described above can be carried out to apply a treatment formulation to a user's skin and to finish the treatment formulation on the user's skin. However, any type of formulation, such as other personal care formulations, can be used as part of the method disclosed above.

It should be noted that for purposes of this disclosure, terminology such as “upper,” “lower,” “vertical,” “horizontal,” “inwardly,” “outwardly,” “inner,” “outer,” “front,” “rear,” etc., should be construed as descriptive and not limiting the scope of the claimed subject matter. Further, the use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

The embodiments of the invention in which an exclusive property or privilege is claimed are defined as follows:

1. An applicator for use in treating a portion of skin, the applicator comprising:
an applicator tip having an end configured to contact the portion of skin, wherein the end of the applicator tip includes a thermally-conductive material;
a sonic source mechanically coupled to the applicator tip;
a heat source thermally coupled to the end of the applicator tip; and
at least one controller configured to control operation of the sonic source and the heat source;
wherein, during operation of the applicator, the at least one controller controls operation of the sonic source to impart mechanical movements to the applicator tip at a sonic frequency and controls operation of the heat source to transfer heat to the end of the applicator tip via the thermally-conductive material of the applicator tip.

2. The applicator of claim 1, wherein the heat source includes a heating disk.

3. The applicator of claim 2, wherein the heating disk includes a plurality of layers of electrical components positioned on an alumina substrate.

4. The applicator of claim 1, further comprising:
a heat sensor configured to generate a signal based on a temperature of the applicator tip and to send the signal to the controller.

5. The applicator of claim 4, wherein the temperature of the applicator tip includes one or more of a temperature of the applicator tip or a temperature of the heat source.

6. The applicator of claim 4, wherein the at least one controller is configured to control the heat source based on the signal sent to the at least one controller by the heat sensor.

7. The applicator of claim 1, wherein the at least one controller is configured to control operation of the heat source based on a target temperature of the end of the applicator tip.

8. The applicator of claim 7, wherein the target temperature is in a range from about 32° C. to about 50° C.

9. The applicator of claim 1, wherein the mechanical movements at the sonic frequency are reciprocating mechanical movements.

10. The applicator of claim 1, wherein the mechanical movements at the sonic frequency are oscillatory mechanical movements.

11. The applicator of claim 1, further comprising:
a contact member around the end of the applicator tip, wherein the contact member is configured to contact the portion of skin, and wherein the contact member and the end of the applicator tip form a concave pocket configured to contain formulation between the end of the applicator tip and the portion of skin.

12. The applicator of claim 1, wherein the end of the applicator tip is formed with a concave shape, and wherein the concave shape forms a pocket configured to contain formulation between the end of the applicator tip and the portion of skin.

13. The applicator of claim 12, wherein the thermally-conductive material forms the concave shape of the end of the applicator tip.

14. The applicator of claim 1, wherein the applicator tip includes a thermally-insulating material, and wherein the thermally-conductive material is embedded within the thermally-insulating material.

15. A method for treating a portion of skin using an applicator that includes an applicator tip, wherein the applicator tip includes an end configured to contact the portion of skin, the method comprising:
activating a sonic source of the applicator, wherein the sonic source is mechanically coupled to the applicator tip;
activating a heat source of the applicator, wherein the heat source is thermally coupled to the end of the applicator tip;
controlling operation of the sonic source to impart mechanical movements to the applicator tip at a sonic frequency; and
controlling operation of the heat source to transfer heat to the end of the applicator tip via a thermally-conductive material of the applicator tip.

16. The method of claim 15, further comprising:
applying a treatment formulation between the end of the applicator tip and the portion of skin during operation of the sonic source and operation of the heat source.

17. The method of claim 16, wherein applying the treatment formulation includes bringing the end of the applicator tip into contact with the treatment formulation when the treatment formulation is in a non-fluid form.

18. The method of claim 17, wherein the operation of the heat source is controlled such that heat transferred to the end of the applicator tip causes the treatment formulation to convert from the non-fluid form to a fluid form when the treatment formulation is in contact with the end of the applicator tip.

19. The method of claim 18, wherein applying the treatment formulation includes applying the treatment formulation in the fluid form to the portion of skin.

20. The method of claim 16, wherein the mechanical movements imparted to the applicator tip at the sonic frequency apply a shear stress to the treatment formulation that causes a viscosity of the treatment formulation to be lowered.
